(19) United States
(12) Patent Application Publication
(10) Pub. No.: US 2014/0243995 A1
(43) Pub. Date: Aug. 28, 2014

(54) STACKED PLANAR SHEET TISSUE ENGINEERING SCAFFOLDS WITH 3D STRUCTURAL ORDER

(71) Applicants: CHARLES STARK DRAPER LABORATORY, INC., Boston, MA (US); THE MASSACHUSETTS INSTITUTE OF TECHNOLOGY, Cambridge, MA (US)

(72) Inventors: Martin E. Kolewe, Chestnut Hill, MA (US); Lisa E. Freed, Lexington, MA (US); Robert Langer, Newton, MA (US); Caprice Gray Haley, Somerville, MA (US)

(73) Assignees: THE MASSACHUSETTS INSTITUTE OF TECHNOLOGY, Cambridge, MA (US); CHARLES STARK DRAPER LABORATORY, INC., Boston, MA (US)

(21) Appl. No.: 13/780,892

(22) Filed: Feb. 28, 2013

(51) Int. Cl. A61L 27/56

(52) U.S. Cl. CPC A61L 27/56
USPC 623/23.72

(57) ABSTRACT

A three-dimensional tissue engineering scaffold device and related methods are disclosed herein. The tissue engineering scaffold includes a plurality of polymers sheets. Each polymer sheet includes a plurality of micro-scale pores defined through the polymer sheet. The polymer sheets of the tissue engineering scaffold are aligned and stacked such that some of the pores of neighboring sheets are offset along at least one axis of the pores. The offset pores create features within the tissue engineering scaffold. In some implementations, the tissue engineering scaffold device is seeded with cells. In certain implementations, the tissue engineering scaffold is either implanted into a patient or used to grow functional tissue ex vivo.

### BACKGROUND OF THE DISCLOSURE

In tissue engineering applications, anisotropy at the micro-scale level is important in creating functional tissue. Cells within tissue are often aligned directionally within specific orthogonal planes. Accordingly, cellular alignment is also important for tissue function in numerous tissue types, such as nerve and heart muscle. Previous scaffold fabrication techniques lack ordered architectures, often having random sponge-like, or felt-like structures.

### SUMMARY OF THE DISCLOSURE

According to one aspect of the disclosure, a three-dimensional tissue engineering scaffold device includes a first polymer sheet having a first plurality of micro-scale pores arranged in a first ordered fashion and a second polymer sheet having a second plurality of micro-scale pores arranged in a second ordered fashion. The second polymer sheet is stacked onto the first polymer sheet such that the first plurality of micro-scale pores are partially aligned with, but laterally offset from, the second plurality of micro-scale pores, such that the first and second plurality of micro-scale pores define paths through the tissue engineering scaffold.

In some implementations, first plurality of micro-scale pores have a first axis that is orthogonal to and longer than a second axis, and the second plurality of micro-scale pores have a third axis that is orthogonal to and longer than a fourth axis. In certain implementations, the length of the micro-scale pores along an axis is between about 10 microns and about 250 microns.

In other implementations, the first axis is parallel to the third axis and the second axis is parallel to the fourth axis. In some implementations, the first plurality of micro-scale pores is laterally offset with respect to the second plurality of pores along at least one of the first and second axes.

In certain implementations, the first plurality of micro-scale pores are laterally offset with respect to the second plurality of pores along the first and third axes and are aligned with the second plurality of pores along the second and fourth axes. In other implementations, the first plurality of micro-scale pores are laterally offset with respect to the second plurality of pores along the second and fourth axes and are aligned with the second plurality of pores along the first and third axes, and in yet other implementations, the first plurality of micro-scale pores are laterally offset with respect to the second plurality of pores along the first and second axes. The offset of the first plurality of micro-scale pores from the second plurality of pores creates at least one feature in an axis orthogonal to the surface of the first and second polymer sheets in some implementations.

In some implementations, a third polymer sheet is coupled to the first and second polymer sheets. The third polymer sheet has a third plurality of micro-scale pores in a third ordered arrangement. The third plurality of micro-scale pores are partially aligned with the second plurality of micro-scale pores such that the first, second, and third pluralities of micro-scale pores form paths through the tissue engineering scaffold.

In certain implementations, the third plurality of micro-scale pores are laterally offset with respect to the second plurality of micro-scale pores along the same axis of the offset between the first plurality of micro-scale pores and the second plurality of micro-scale pores. The direction of the offset between the second and third plurality of micro-scale pores along the first axis is opposite to the direction of offset along of the first axis of the second plurality of micro-scale pores with respect to the first plurality of micro-scale pores in certain implementations.

In some implementations, the direction of the offset between the second and third plurality of micro-scale pores along the first axis is the same direction of the offset along of the first axis of the second plurality of micro-scale pores with respect to the first plurality of micro-scale pores, and in other implementations, the third plurality of micro-scale pores are laterally offset with respect to the second plurality of micro-scale pores along a different axis than the first plurality of micro-scale pores are offset from the second plurality of micro-scale pores.

In yet other implementations, cells are seeded in the tissue engineering scaffold, such as in the paths through the tissue engineering scaffold. In some implementations, the cells include at least one of cardioprogenitor cells, cardiac muscle cells; cardiac fibroblasts; endothelial cells; skeletal muscle cells; smooth muscle cells; endothelial progenitor cells; skeletal muscle progenitor cells; neuroprogenitor cells; nerve cells; dermal fibroblasts; ectodermal cells; bone cells; cartilage cells; tendon cells; ligament cells; hepatocytes; pancreatic islet cells; intestinal cells; progenitor cells derived from a tissue selected from the group consisting of bone marrow or fat; induced pluripotent stem cells (iPS cells); and genetically transformed cells.

In some implementations, the cells are seeded in the tissue engineering scaffold such that they align parallel to the aligned axes of the micro-scale pores and weave above the offset axes of the first polymer sheet and below the offset axes of the second polymer sheet. In some implementations, the cells stay predominantly in the same plane as the polymer sheets.

In some implementations, the polymer sheets of the tissue engineering scaffold have a height between about 10 microns and about 150 microns, and include elastomeric degradable polymers such as poly(glycerol sebacate). In certain implementations, the polymer sheets degrade upon exposure to water, heat, enzymes, or UV light. In other implementations, the polymer sheets are treated with an agent. The agent can be at least one of solubilized extracellular matrix, collagen, fibronectin, laminin, elastin, an agent that promotes cell adhesion, a cellular growth and/or cell differentiation promoter, a fibrosis and/or microbial growth inhibitor, a polymer sheet degradation inhibitor, and a polymer sheet degradation promoter.

In certain implementations, the pores of the polymer sheets are rectangular, circular, square, or any combination thereof. The porosity of the polymer sheets is about 60% in some implementations.

According to another aspect of the disclosure, a method for manufacturing a three-dimensional tissue engineering scaffold device includes providing a first polymer sheet having a first plurality of micro-scale pores defined therethrough and arranged in a first ordered fashion. The method also includes providing a second polymer sheet having a second plurality of micro-scale pores defined therethrough and arranged in a second ordered fashion. The second polymer sheet is stacked onto the first polymer sheet such that the first plurality of micro-scale pores are partially aligned with, but are laterally offset from, the second plurality of micro-scale pores. The first and second pluralities of micro-scale pores define paths through the tissue engineering scaffold. The method also includes, bonding the second polymer sheet to the first polymer sheet.

In some implementations, the first plurality of micro-scale pores have a first axis that is orthogonal to and longer than a second axis, and the second plurality of micro-scale pores have a third axis that is orthogonal to and longer than a fourth axis. In certain implementations, the length of the pores along an axis is between about 10 microns and about 250 microns.

In some implementations, the method includes laterally offsetting the first plurality of micro-scale pores with respect to the second plurality of pores along at least one of the first and second axes. The lateral offset includes offsetting the first plurality of micro-scale pores with respect to the second plurality of pores along the first and third axes and are aligned with the second plurality of pores along the second and fourth axes in some implementations.

In certain implementations, the method also includes laterally offsetting the first plurality of micro-scale pores with respect to the second plurality of pores along the second and fourth axes and aligning the pores along the first and third axes. In other implementations, the method further includes laterally offsetting the first plurality of micro-scale pores with respect to the second plurality of pores along the first and second axes.

In some implementations the method includes, providing a third polymer sheet having a third plurality of micro-scale pores defined therethrough and arranged in a third ordered fashion and then coupling the third polymer sheet to the first and second polymer sheets such that the third plurality of micro-scale pores are partially aligned with the second plurality of micro-scale pores causing the first, second, and third pluralities of micro-scale pores to form paths through the tissue engineering scaffold. In certain implementations, coupling one polymer sheet to another polymer sheet includes applying heat and pressure to the polymer sheets.

In some implementations, the polymer sheets are formed on a sacrificial layer atop a substrate and then removed from the substrate by dissolving the sacrificial layer. In certain implementations, the sacrificial layer includes maltose. In some implementations of the method, the polymer sheets include poly(glycerol sebacate).

In certain implementations, the micro-scale pores are created using one of photolithography, injection molding, hot embossing, and deep reactive ion etching, melt-casting, spin-coating, solid freeform fabrication, and laser micro-ablation.

In other implementations, the method also includes seeding cells in the paths of the tissue engineering scaffold. The cells include at least one of cardioprogenitor cells, cardiac muscle cells; cardiac fibroblasts; endothelial cells; skeletal muscle cells; smooth muscle cells; endothelial progenitor cells; skeletal muscle progenitor cells; neuroprogenitor cells; nerve cells; dermal fibroblasts; ectodermal cells; bone cells; cartilage cells; tendon cells; ligament cells; hepatocytes; pancreatic islet cells; intestinal cells; progenitor cells derived from a tissue selected from the group consisting of bone marrow or fat; induced pluripotent stem cells (iPS cells); and genetically transformed cells.

In certain implementations, cells in the tissue engineering scaffold are seeded such that they align parallel to the aligned axes of the micro-scale pores and weave above the offset axes of the first polymer sheet and below the offset axes of the second polymer sheet, such that the cells stay predominantly in the same plane as the polymer sheets. In other implementations, the polymer sheets are treated with an agent. The agent can be at least one of solubilized extracellular matrix, collagen, fibronectin, laminin, elastin, an agent that promotes cell adhesion, a cellular growth and/or cell differentiation promoter, a fibrosis and/or microbial growth inhibitor, a polymer sheet degradation inhibitor, and a polymer sheet degradation promoter.

According to yet another aspect of the disclosure, a method of treatment includes implanting a tissue engineering scaffold into a patient. The tissue engineering scaffold includes a first polymer sheet having a first plurality of micro-scale pores defined therethrough and arranged in a first ordered fashion and a second polymer sheet having a second plurality of micro-scale pores arranged therethrough and arranged in a second ordered fashion, wherein the second polymer sheet is stacked onto the first polymer sheet such that the first plurality of micro-scale pores are partially aligned with, but are laterally offset from, the second plurality of micro-scale pores, such that the first and second plurality of micro-scale pores define paths through the tissue engineering scaffold.

In certain implementations, the method also includes seeding the tissue engineering scaffold with cells prior to implantation, and in some implementations the cells are harvested from the patient.

### BRIEF DESCRIPTION OF THE DRAWINGS

The skilled artisan will understand that the figures, described herein, are for illustrative purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1A is an isometric view of an example porous polymer sheet.

FIGS. 1B-1F are top views of example polymer sheets, similar to that of FIG. 1A, with different pores configurations.

FIGS. 2A-2C are solid models of example polymer tissue engineering scaffolds illustrating different stacking configurations using polymer sheets similar to those shown in FIG. 1.

FIGS. 3A-3D show cross sectional views of example offset configurations for scaffolds consisting of three polymer sheets, such as those of FIG. 1.

FIG. 4 is a top view of an example polymer tissue engineering scaffold including a plurality of pore types and at least two polymer sheets.

FIGS. 5A-5F are a series of cross sectional diagrams illustrating an example process for fabricating a polymer sheet similar to the polymer sheets of FIGS. 1A-1F.

FIG. 6 is a flow chart of an example method for using a polymer tissue engineering scaffold.

FIG. 7 is a flow chart of an example method for manufacturing a polymer tissue engineering scaffold.

FIG. 8 shows a series of example models and micrographs of two layer polymer tissue engineering scaffolds, similar to those shown in FIGS. 2A and 2B.

FIG. 9 is a graph representing the effective stiffness of the two layer polymer tissue engineering scaffolds from FIGS. 8A-8D.

FIGS. 10A and 10B are confocal micrographs of the two layer polymer tissue engineering scaffolds of FIG. 8 seeded with heart cells.

FIG. 10C is a confocal micrograph of native cardiac tissue, for comparison with the micrographs shown in FIGS. 10A and 10B.

FIG. 10D is a side view of a solid model representative of the polymer tissue engineering scaffolds shown in FIG. 10B, further illustrating the path the seeded cells grow through the polymer tissue engineering scaffold.

FIG. 11 is a series of micrographs of the two layer polymer tissue engineering scaffolds shown in FIG. 8 with cultured muscle cells.

FIG. 12 is a plot of cellular alignment, as determined by Fast Fourier Transform (FFT) analysis, of the cells seeded in the scaffolds of FIG. 11, in accordance with an implementation of the present disclosure.

FIGS. 13A and 13B illustrate possible positions of scaffolds when used as epicardial grafts that provide anisotropic mechanical restraint and alignment for cells.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The method and systems disclosed herein provide for the scaling-up of polymer scaffolds, which is useful for furthering an understanding of cells' interactions with geometric and topographic cues in their three-dimensional microenvironment. Additionally, the system and methods provide a practical method to produce the scaled-up, functional tissue constructs which is adaptable to a higher-throughput process flow more suitable for larger scale manufacturing than previous MEMS fabrication approaches.

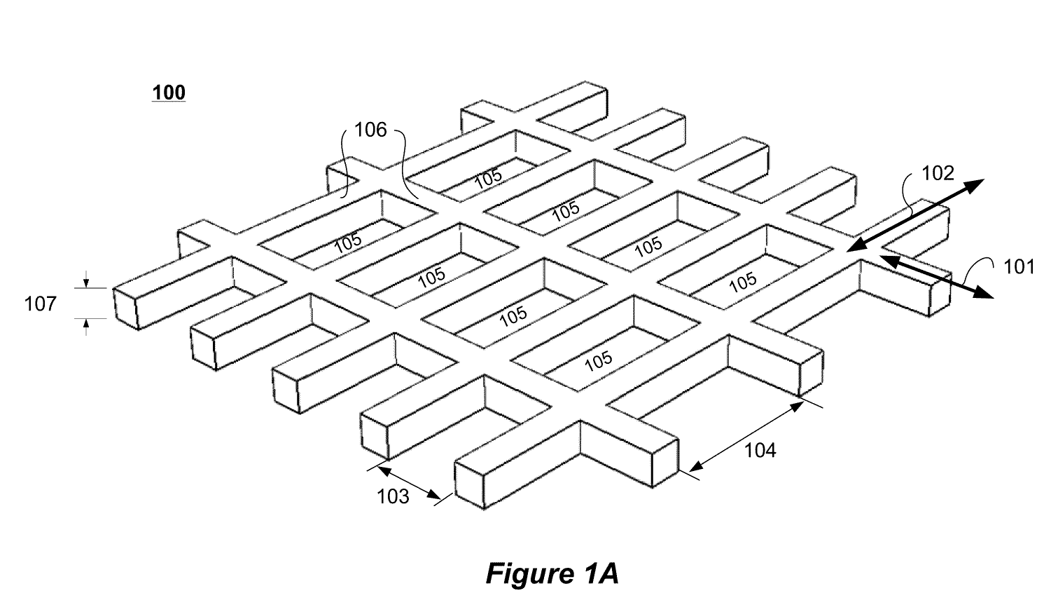

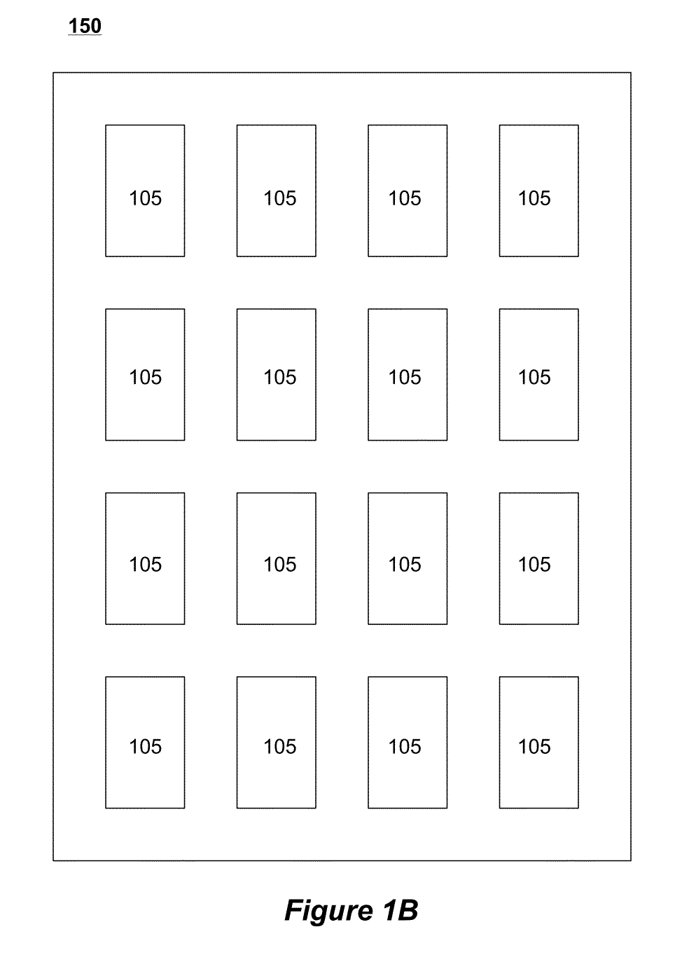

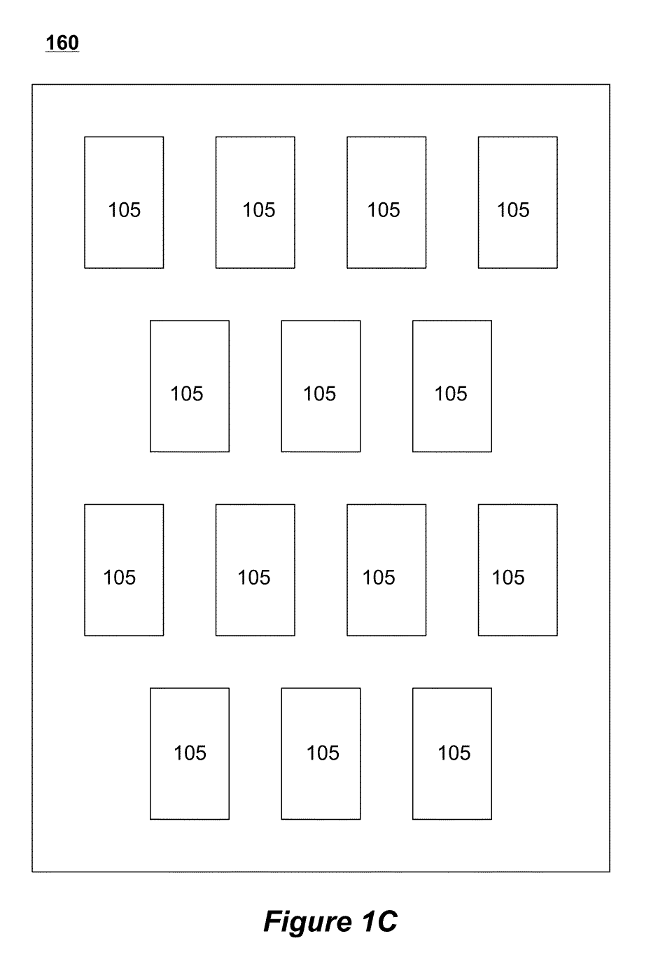

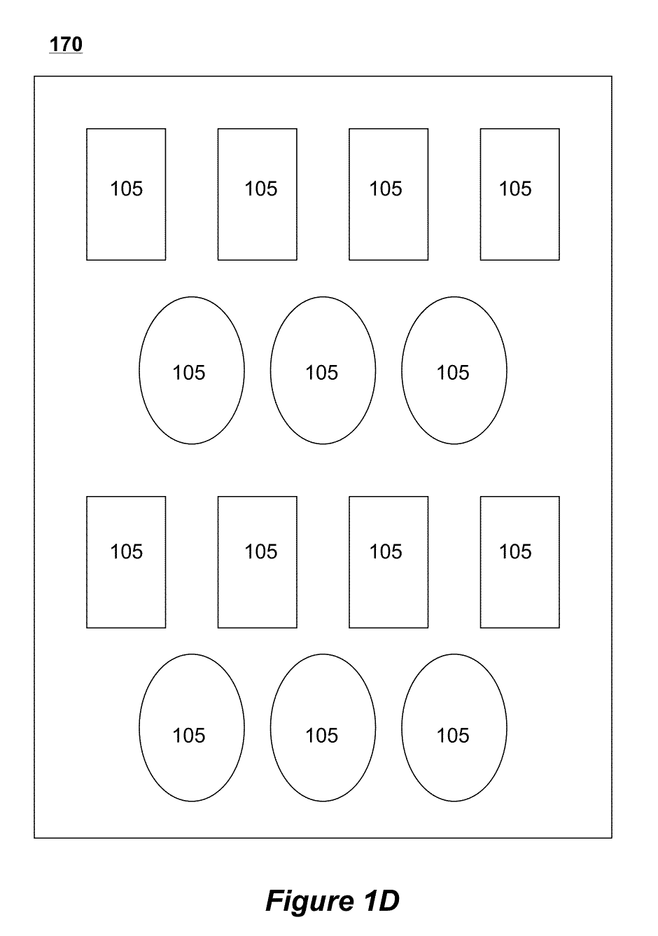

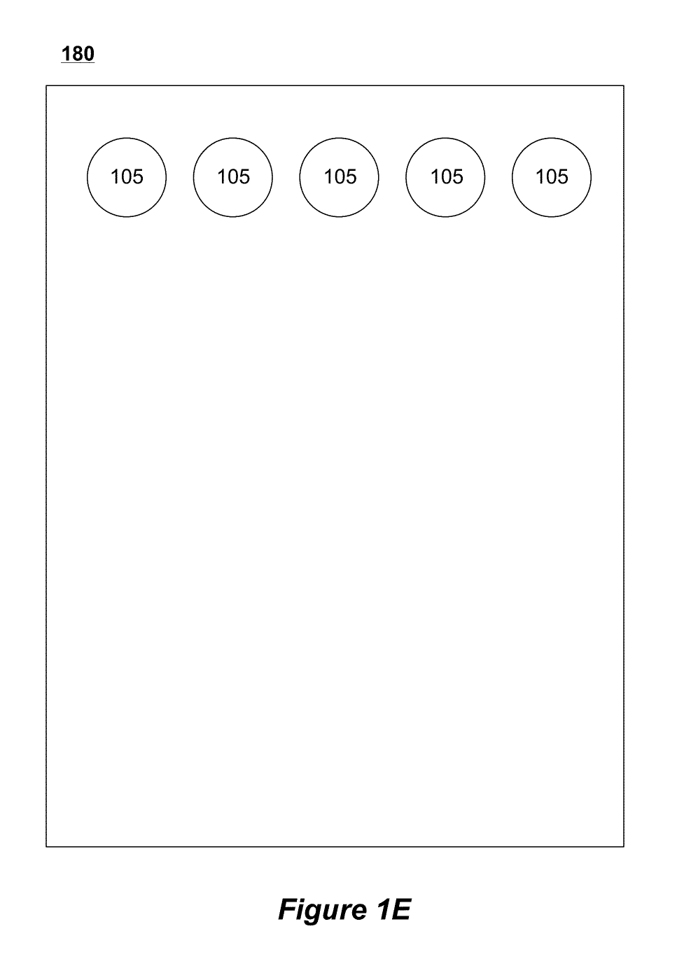

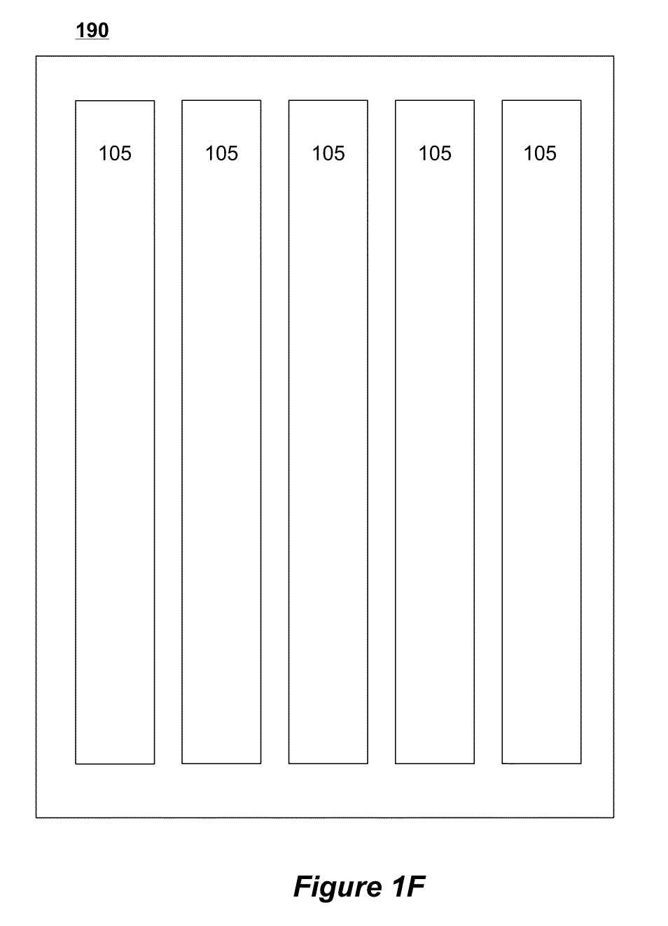

FIG. 1A is an isometric view of a single layer polymer sheet 100, which can be used in a three dimensional polymer scaffold. The polymer sheet 100 includes a plurality of pores 105. Each pore 105 includes a first axis 101 and a second axis 102. A length is associated with each axis of the pore 105. In the illustration of the polymer sheet 100, the length of the pore 105 along the first axis 101 is referred to as the width 103 of the pore 105, and the length of the pore 105 along the second axis 102 is referred to as height 104 of the pore 105, additionally each polymer sheet 100 has a specific thickness 107. The solid polymer material separating the pores in the polymer sheet 100 is referred to as a strut 106.

The single layer polymer sheet 100 represents the basic building block of a porous polymer scaffold, also referred to as a tissue engineering scaffold. As discussed below, a plurality of polymer sheets 100 are stacked upon one another to create a multi-layered polymer scaffold with three-dimensional features. In some implementations, the polymer sheet 100 is created from a biocompatible and/or biodegradable polymer. Example polymers include, but are not limited, to poly(glycerol sebacate) (PGS), polyamides, poly(amino acids), polyanhydrides, polycaprolactones, polydioxanones, polyesters, polyesteramides, polyorthoesters, polyphosphazenes, polyacetals, chitin, chitosan, collagen, methacrylated gelatin, polycarbonates, poly(dimethyl siloxane) (PDMS), polyhydroxy-butyrates, polyurethanes, and copolymers, terpolymers, or any combination thereof. In some implementations, in place of a polymer, the sheet is constructed from a slice of biological tissue and/or its extracellular matrix. For example, a microtome can be used to create a 100 μm slice of biological tissue into which the below described pores are created.

The polymer sheet 100, as illustrated, includes a plurality of pores 105. Pores 105 can also be referred to as micro-pores. In some implementations, the pores are created in the polymer sheet 100 by micro-machining, laser micro-ablation, micro-molding off etched silicon wafers, injection molding, inkjet printing, direct laser writing, solid free form fabrication, photolithography, hot embossing, deep reactive ion etching, and melt-casting, or any combinations thereof. In some implementations, the width 103 and height 104 of a pore 105 are equal. In other implementations, the width 103 and height 104 of a pore 105 are not equal. As illustrated in FIG. 1, the height 104 is greater than the width 103. In some implementations, the width 103 and height 104 are between about 50 μm and about 200 μm or about 200 μm and 500 μm. In yet other implementations, the ratio of the width 103 to the height 104 can be one of 1:1, 1:2, 1:3, 1:4, 1:5, and 1:10. In some implementations, the struts 106 between the pores 105 are about 20 to 50 μm wide. In yet other implementations, the spacing and size of the pores 105 is defined such that the polymer sheet 100 has a given porosity. For example, the porosity may be selected to be between one of about 30% to about 50%, and between about 50% and about 85%. In some implementations, the polymer sheet 100 is created to have a thickness of between about 50 μm and about 100 μm or between about 100 μm and about 250 μm.

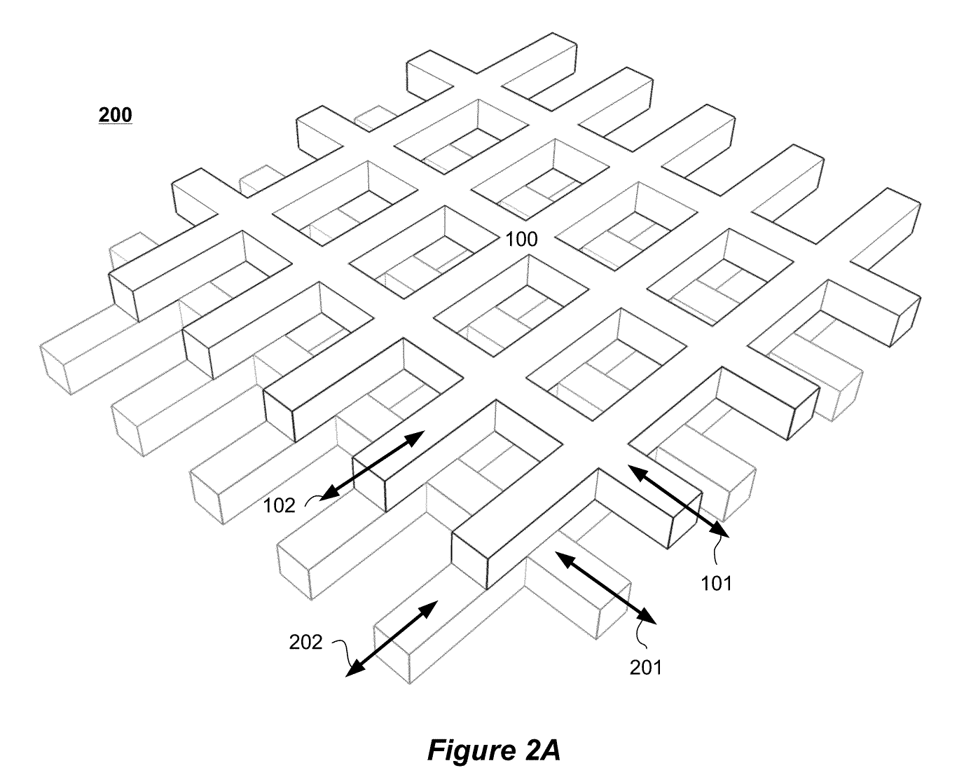

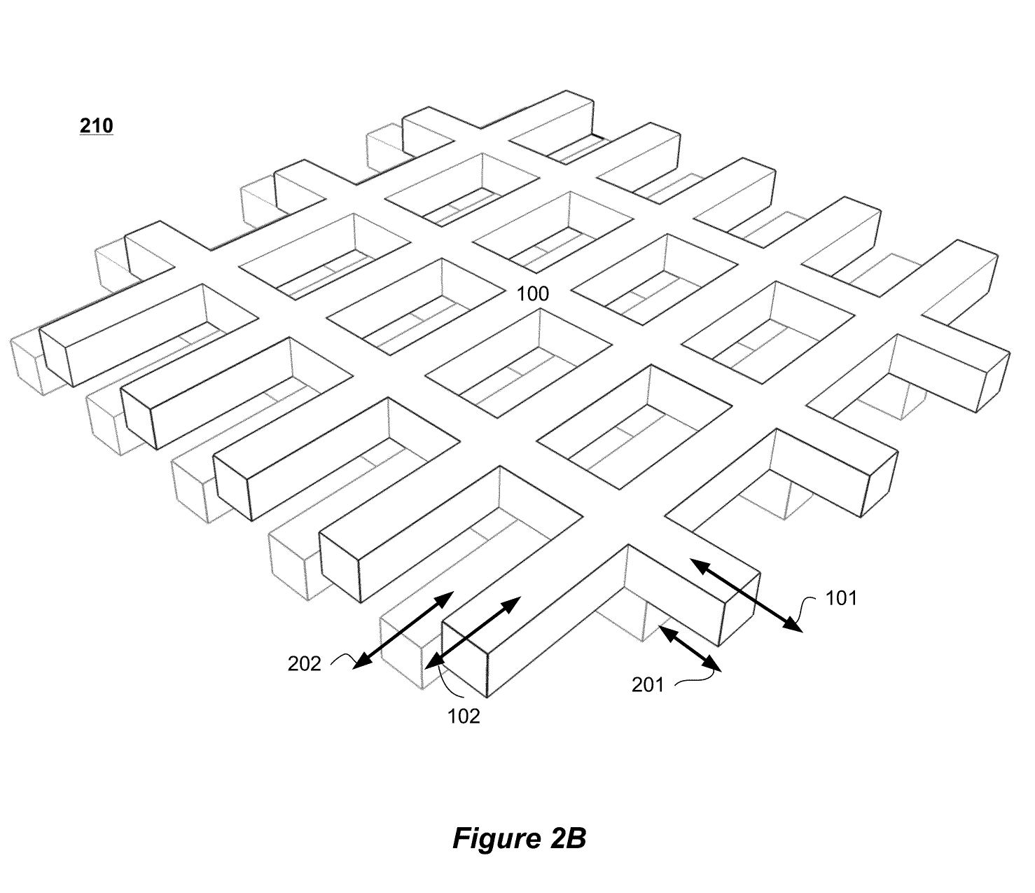

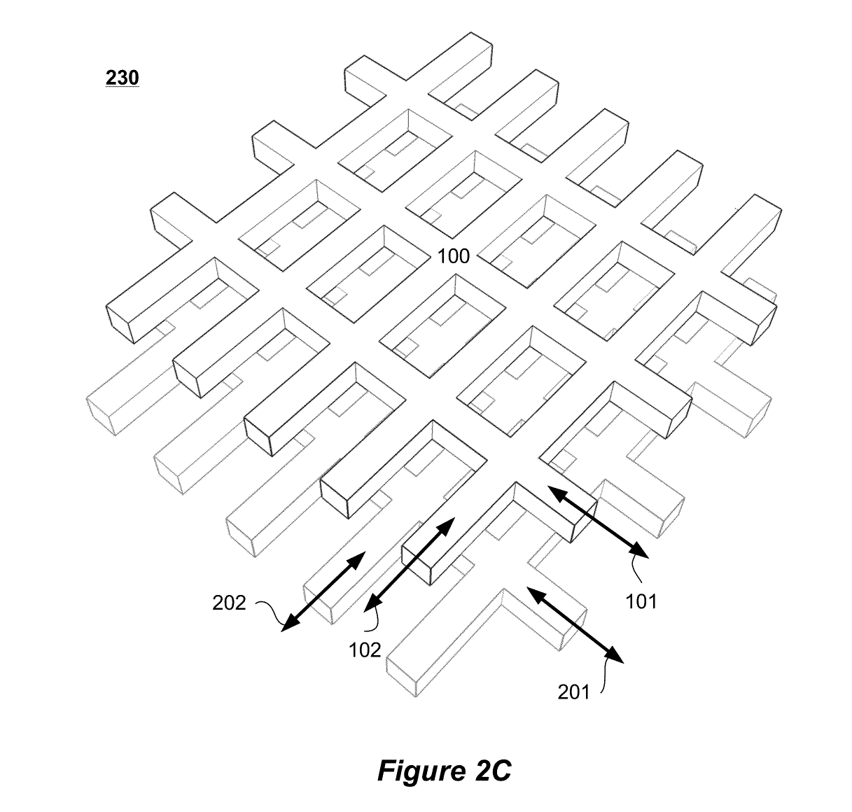

FIG. 1B illustrates an alternative polymer sheet 150 implementation having an enclosed periphery. As illustrated, the pores 105 of the polymer sheet 150 are all within the periphery of the polymer sheet 150. As discussed below in FIG. 2, in some implementations more than one polymer sheet is aligned with respect to one or more struts 106. In other implementations, more than one polymer sheet is aligned based on the relative positions of their peripheries or borders. In some implementations, the straight edges of the peripheries of the polymer sheet 150 facilitate this type of alignment. In some of these implementations, the pore offset is created by creating the pores such that they are offset from one another when the peripheries or borders of the more than one polymer sheets 100 are aligned.

FIG. 1C illustrates a polymer sheet 160 with offset pore rows. In some implementations, such as the implementation of the polymer sheet 100, the pores 105 within the sheet are aligned along both the first axis 101 and the second axis 102. In contrast, the polymer sheet 160 includes offset rows of pores.

In some implementations, the offset of the pores 105, the sizes of the pores 105, and shapes of the pores 105 are configured such that alignment of more than one polymer sheets with respect to one or more pore axes creates three-dimensional scaffolds with three-dimensional architectural order that facilitates the development of engineered tissues with three-dimensional architectural order. In some implementations, the three-dimensional architectural order of the engineered tissue mimics naturally occurring tissue. Polymer sheet 170 of FIG. 1D, illustrates a polymer sheet that includes a plurality of different pore shapes and arrangements. Polymer sheet 170 includes elliptical pores 171 offset from rectangular pores 105.

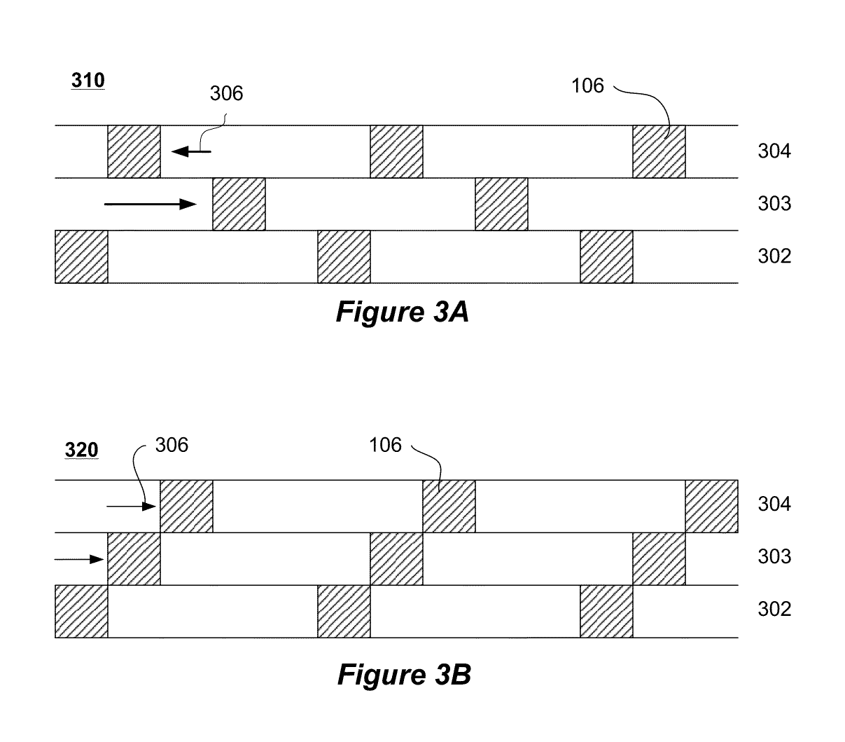

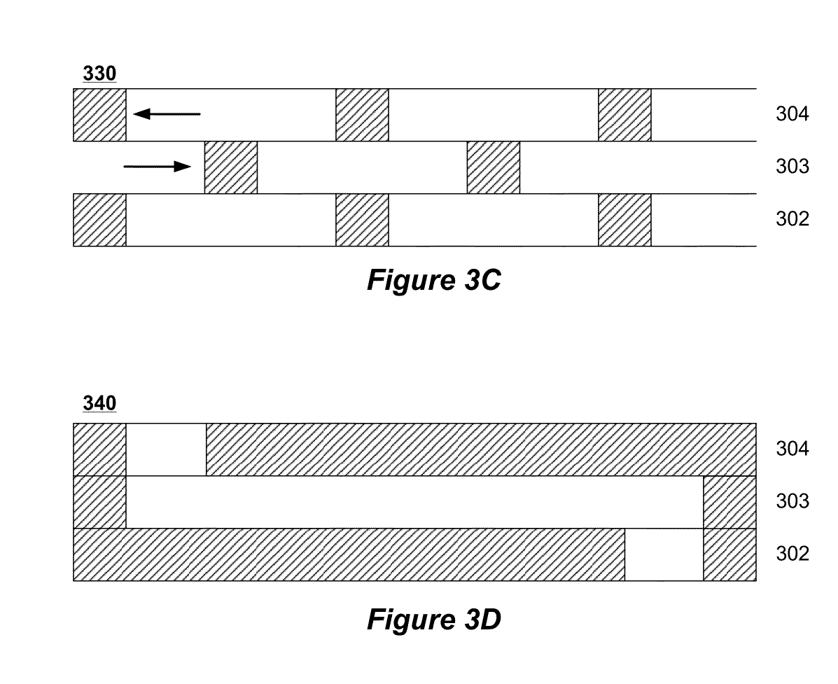

FIG. 1E illustrates a polymer sheet 180 with a plurality of circular pores, and FIG. 1F illustrates a polymer sheet 190 with a plurality of high aspect ratio rectangular pores. As illustrated in FIG. 3D, in some implementations, polymer sheet 190 is sandwiched between two copies of polymer sheets 180 to form multi-layered scaffolds with perusable microchannels forming fluidic inlets in the first layer, microchannels in the second layer, and fluidic outlets in the third layer.

FIGS. 2A-2C illustrate a plurality of different offset configurations between different layers of the polymer sheets 100. To simplify the illustration, FIGS. 2A-C show only two polymer sheets 100; however, one skilled in the art would recognize that more than two polymer sheets 100 may be stacked upon one another to create a polymer scaffold.

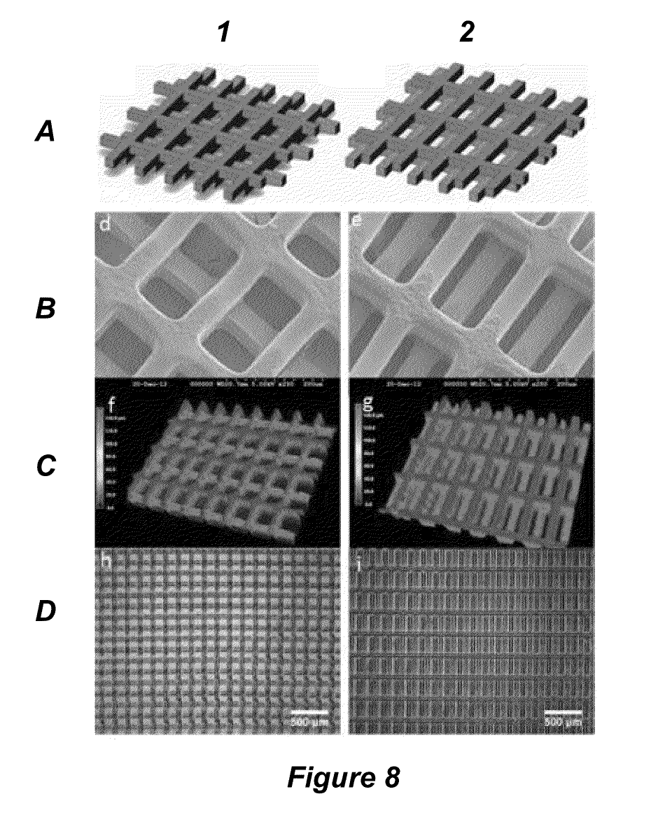

FIG. 2A illustrates a short strut offset stacking arrangement 200. As in FIG. 1, the short struts of the pores are included in the first axis 101 of the pores. Accordingly, the short strut offset stacking arrangement 200 may also be referred to as a first axis offset stacking arrangement in these examples. In the short strut offset arrangement 200, the short struts between the pores are offset from one another, while the long struts between the pores remained aligned along the second axis. As illustrated below in reference to FIG. 8, if the struts 106 have the same width in the first and second axes, a short strut offset creates a multi-layered scaffold with greater porosity in the direction orthogonal to the face of the polymer sheets 100 when compared to a long strut offset.

Similar to FIG. 2A, FIG. 2B illustrates two stacked polymer sheets 100. The polymer sheets 100 of FIG. 2B are stacked with a long strut offset arrangement 210. In this arrangement, the short struts of the polymer sheets 100 remain aligned along the first axis, while the long struts of the polymer sheets 100 are offset along the second axis 102.

FIG. 2C illustrates two polymer sheets 100 that are offset along both the first axis 101 and the second axis 102. In this arrangement, neither the first nor second axis is aligned.

In some implementations, the polymer scaffold is created by the stacking of more than two polymer sheets 100. In these implementations, the additional polymer sheets 100 can also be offset along their first axis 101 and/or second axis 102. When stacking and offsetting the polymer sheets 100, a number of stacking parameters can be altered to create polymer scaffolds with different three dimensional architectural features. These stacking parameters can include, but are not limited to, the direction which a first polymer sheet 100 is offset from a second polymer sheet 100: the distance a first polymer sheet 100 is offset from a second polymer sheet 100; the number of offset axes; the pore design, shape, and/or size in each layer; or any combination thereof.

FIGS. 3A-3D illustrate the relationship of stacking parameters in relation to stacking direction and stacking distance. FIG. 3A illustrates a cross-sectional side view of a polymer scaffold 310. Struts 106 are indicated as the shaded regions of the layer. As illustrated, the polymer scaffold 310 includes an alternating offset direction. Additionally, the polymer scaffold 310 also includes offsets of varying magnitude. Arrows 306 indicate the direction and magnitude of the offset. Layer 303 is offset from layer 302 in a first direction, and layer 304 is offset from layer 303 in a second direction. Layer 303 of the polymer scaffold 310 is offset from layer 302 by a greater distance than layer 304 is offset from layer 303. A person of ordinary skilled in the art will appreciate that for a polymer scaffold of significant size the same alternating direction offset pattern can also be created by offsetting the layers a distance greater than half the distance between adjacent struts 106. For example, the polymer scaffold 310 can also be viewed as a polymer scaffold containing a large, single direction offset. In some implementations, the offset magnitude is set to allow seeded cells to elongate and weave in between the polymer layers. In some implementations, the offset length is between about 10 μm and about 100 μm.

FIG. 3B illustrates a polymer scaffold 320 that includes a plurality of layers 302, 303, and 304, each having a single offset distance and single offset direction. In some implementations, this offset design may be used to route cells into a new plane.

FIG. 3C illustrates a polymer scaffold 330 that includes a plurality of layers 302, 303, and 304, each having an offset size of half the distance between neighboring struts. As illustrated, an offset size of half the pore length causes the struts 106 of layer 304 to be directly above the struts 106 of layer 302. Similar to FIG. 3A, in some implementations, offset of polymer scaffold 330 allows seeded cells to elongate and weave in between the polymer layers.

FIG. 3D illustrates a polymer scaffold 340 that includes a plurality of layers 302, 303, and 304. In this example, pores in layers 304 and 302 are connected by an elongated pores in layer 303. Thus, as illustrated, polymer scaffold 340 produces microchannel in layer 303, with fluidic inlets and outlets in layers 302 and 304. In some implementations, as the polymer scaffold increases in size, channels, as illustrated in polymer scaffold 340, are employed to perfuse the seeded cells with adequate nourishment.

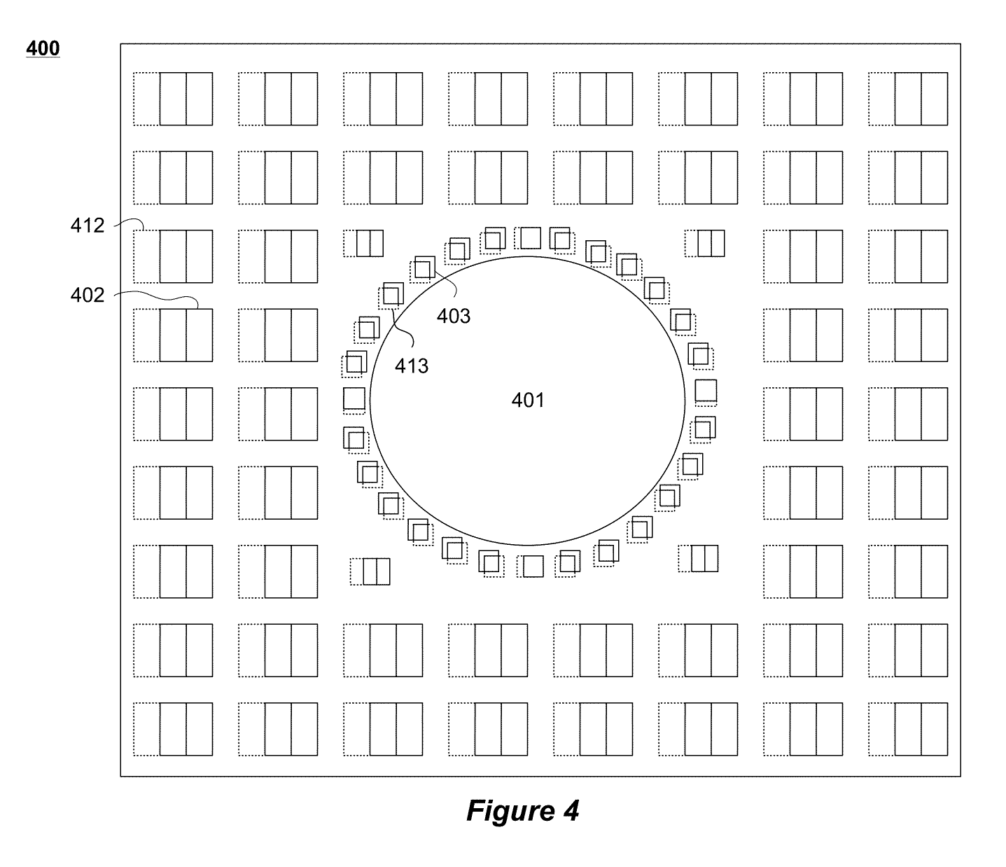

FIG. 4 is a top view illustration of a multi-layered polymer scaffold 400 having a plurality of pores and pore configurations. In some implementations, the polymer scaffolds disclosed herein are seeded with cells to grow a single type of tissue. For example, the scaffold may be seeded with cardioprogenitor cells to grow tissue for the repair of the myocardium. In some implementations, a tissue includes a plurality of cell types. Accordingly, in some of these implementations, the polymer scaffolds is seeded with a plurality of cells types. For example, in creating myocardium tissue, the polymer scaffold may be seeded with muscle cells, fibroblasts, and endothelial cells. In some implementations, the polymer scaffold is seeded with cells to create a plurality of tissue types.

In each layer, the polymer scaffold 400 includes a plurality of pore shapes and sizes, and is an example of a polymer scaffold that may support the growth of multiple cell and/or tissue types. As illustrated, polymer scaffold 400 includes two polymer sheets. The pores of the first polymer sheet are illustrated with solid lines and portions of the pores in the second polymer sheet that are obscured by the first polymer sheet are indicated as dashed lines.

The polymer scaffold 400 can be used, for example, to grow an endothelial cell-lined lumen surrounded by heart muscle cells. In this example, each layer includes a central pore 401. Each central pore 401 is substantially aligned with the central pore 401 of the layer immediately above and below, creating a central lumen. The central pore 401 is encircled with a first series of pores 403. Pores 403 can be seeded with endothelial cells, such that they eventually form a lining on the wall of the lumen. Surrounding the central pore 401 and pores 403, are a second series of pores 402. Furthering the example, pores 402 can be seeded with parenchymal cells that will develop into the tissue that surrounds the lumen 401. For example, the cells seeded into pores 402 can be muscle cells. In some implementations, the polymer is biodegradable, such that when the polymer degrades the endothelial cells come into direct contact with any fluid present in the lumen, and with muscle cells present in the parenchymal space, mimicking the architecture of natural vascularized muscle tissue.

As briefly discussed above, the polymer scaffold 400 illustrates that in some implementations the borders of each layer of the polymer scaffold are aligned and a porous pattern is created by offsetting the patterning of the pores from layer to layer. In certain implementations, such as those referenced in relation to FIG. 2, the polymer sheets have identical micro-scale features and the offset between each layer's micro-scale features is created by offsetting each layer. Accordingly, the portions of the layers that overhangs a usable portion of the polymer scaffold (for example, when the borders of the various layers making up the scaffold are not aligned during manufacture) may be trimmed and removed. However, in other implementations, such as that of polymer scaffold 400, each layer of the polymer scaffold may not be identical to the layer above or below. In these implementations, the pore offset between each layer may be specifically machined for its position within the polymer scaffold. For example, a first layer of polymer scaffold 400 may have its central pore 401 in the center of the polymer sheet and the other pores arranged about the central pore 401 in a first configuration. To create the effect of a central pore 401 throughout the polymer scaffold, the second polymer sheet cannot simply be offset from the first polymer sheet, as this would create an offset central pore 401. Rather, the second polymer sheet is fully aligned with the first polymer sheet, and the pores of each polymer sheet are machined to produce the desired combination of offset and alignment of the pores in the multi-layered polymer scaffold. The polymer scaffold 400 illustrates that the second polymer sheet is aligned with the first polymer sheet; however, the pores 413 and 412 are shifted from their first layer counterparts 403 and 402.

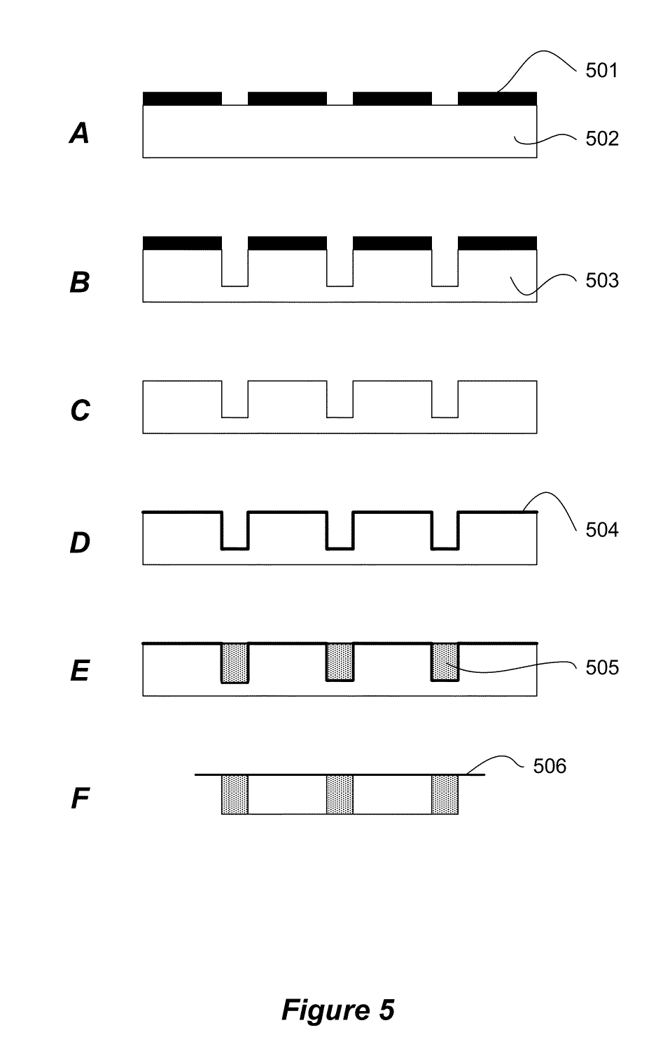

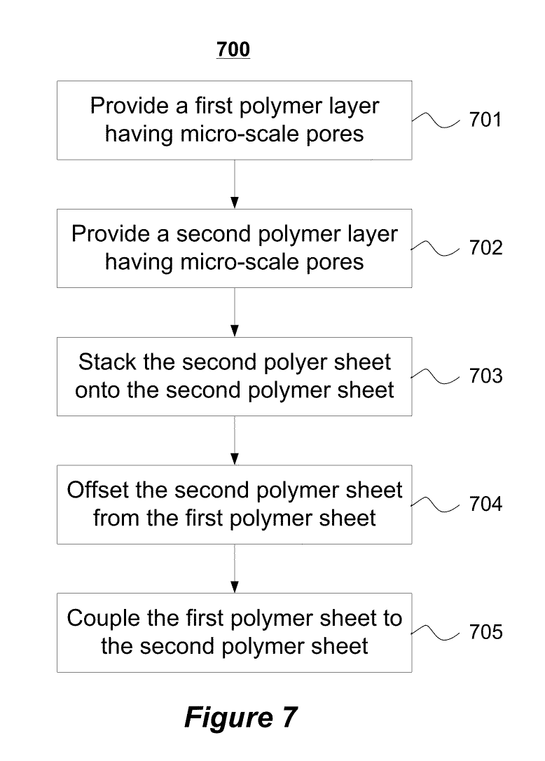

FIG. 5 illustrates a process 500 of manufacturing a polymer sheet 100 with a plurality of pores 105. As discussed below in relation to FIG. 7, in some implementations, a polymer scaffold is created by manufacturing a plurality of polymer sheets 100, which are then aligned and stacked to form the polymer scaffold. In some implementations, by creating large polymer sheets 100, each with a plurality of pores 105, large polymer scaffolds can be created quickly. Additionally, the method described herein allows for the use of rigid, biodegradable elastomeric materials, such as PGS, poly(1,3-diamino-2-hydroxypropane-co-polyol sebacate) (APS), and other materials described above. In some implementations, the process 500 allows for precision assembly of scaffolds constructed with materials desired by the healthcare industry such as implantable polymers including porous, elastomeric, and biodegradable materials.

FIGS. 5A-5F illustrate a series of cross sectional diagrams of the process 500 of manufacturing a polymer sheet. First, in FIG. 5A, a mask 501 is applied to a silicon (Si) wafer 502. Next, in FIG. 5B, the portions of the Si wafer 502 not protected by the mask 501 are etched away using reactive ion etching. The etching leaves channels in the Si wafer 502 that can be used as a mold for the polymer sheet 100. Before being used as a mold, in FIG. 5C, the mask 501 is removed and the Si wafer 502 is cleaned. Next, in FIG. 5D, a sacrificial layer 504 is applied to the etched Si wafer 502. In some implementations, the sacrificial layer 504 is applied by spin coating a sacrificial layer 504 onto the Si wafer 502. The sacrificial layer 504 may be any material that is easily dissolvable. In certain implementations, the sacrificial layer 504 includes a sugar such as maltose. Then, in FIG. 5E, a liquid pre-polymer 505 is cast into the mold. The liquid pre-polymer is cured under heat and vacuum. Once cured, in some implementations, a Teflon backing 506 is applied to the polymer sheet 100 to allow later stacking of the polymer sheets 100. The cured polymer sheet is removed from the Si wafer 502 by dissolving the sacrificial layer 504. In some implementations, the sacrificial layer 504 can be dissolved with water and/or heat. In some implementations, polymer sheets 100 with more than one pore design are molded on a single Si wafer. For example, a Si wafer can be etched such that the different polymer sheets represented by the configurations shown in FIG. 1 are all molded on a single SI wafer.

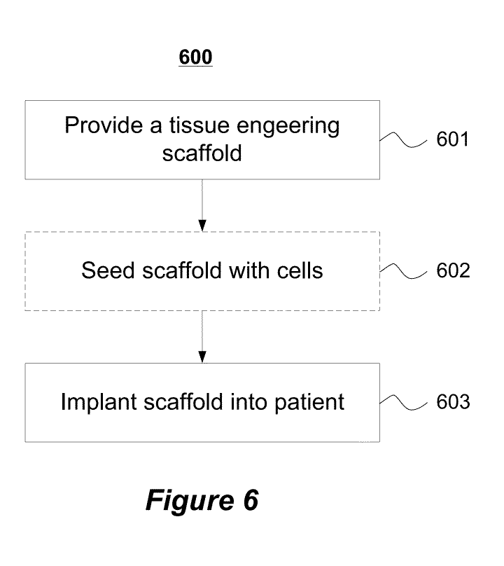

FIG. 6 is a flow chart of a method 600 for implanting a polymer scaffold into a patient. Method 600 provides a general overview of a use of a polymer scaffold according to one implementation of the disclosure. Further details relating to method 600 are described in relation to FIGS. 7 and in reference to the above description of FIG. 5.

The method 600 of implanting a polymer scaffold into a patient begins by providing a polymer scaffold (step 601). Next, the polymer scaffold is seeded with cells (step 602). Then the polymer scaffold is implanted into a patient (step 603).

As set forth above, method 600 begins by providing a polymer scaffold. The construction of a polymer scaffold is discussed in greater detail in relation to FIG. 7. In some implementations, the polymer scaffold is pre-fabricated. In certain implementations, the polymer scaffold is sterilized prior to being seeded with cells and/or implanted. In some implementations, the polymer scaffold is created specifically for the patient into whom it will be implanted. For example, the polymer scaffold may be created in the size and shape required to repair a congenitally defective tissue or a tissue damaged or removed due to disease. In some implementations, the mechanical properties of the polymer scaffold may be designed to match the mechanical properties of the tissue to be repaired.

After obtaining a polymer scaffold, the method 600 continues by seeding the polymer scaffold with cells (step 602). In some implementations, step 602 is an optional step (as indicated by the dashed box in FIG. 6). As discussed above, and in greater detail in relation to the examples below, in some implementations, a plurality of cell types is seeded into the polymer scaffold. In certain implementations only one cell type is seeded into the polymer scaffold. After seeding cells into the polymer scaffold, in some implementations, the cells are cultured for a specific amount of time in an incubator before implanting the cell-seeded polymer scaffold, such that the graft matches the architectural properties of the natural tissue to be repaired. In other implementations, the seeded cells are harvested from the patient prior to implantation of the polymer scaffold. The seeded cells can include, but are not limited to, cardioprogenitor cells, cardiac muscle cells; cardiac fibroblasts; endothelial cells; skeletal muscle cells; smooth muscle cells; endothelial progenitor cells; skeletal muscle progenitor cells; neuroprogenitor cells; nerve cells; dermal fibroblasts; ectodermal cells; bone cells; cartilage cells; tendon cells; ligament cells; hepatocytes; pancreatic islet cells; intestinal cells; progenitor cells derived from a tissue selected from the group consisting of bone marrow or fat; induced pluripotent stem cells (iPS cells); and genetically transformed cells. In some implementations, the polymer scaffold is coated with an agent prior to cellular seeding. The agent can include, but is not limited to a growth medium, solubilized extracellular matrix (ECM) or molecular derivative thereof, collagen, fibronectin, laminin, elastin, an agent that promotes cell adhesion, a cellular growth and/or cell differentiation promoter, a fibrosis and/or microbial growth inhibitor, a polymer sheet degradation inhibitor, and a polymer sheet degradation promoter, or any combination thereof.

Responsive to seeding the polymer scaffold with cells, the polymer scaffold is implanted into the patient (step 603). In certain implementations, the polymer scaffold is not seeded with cells prior to implantation, such that the scaffold provides mechanical support, as is, and/or allows the patient's own cells and/or tissues to grow into the polymer scaffold after implantation.

In yet other implantations, the polymer scaffold is not implanted into a patient, but seeded with cells and used to grow tissue ex vivo. In some implementations, the ex vivo tissue is used to test drugs' and other agents' efficacy and safety.

FIG. 7 is a flow chart of an example method 700 for constructing a polymer scaffold. Method 700 provides further details that can, for instance, be used in step 601 of method 600. The method 700 begins by providing a first polymer sheet (step 701) and provides a second polymer sheet (step 702). The second polymer sheet is then offset from the first polymer sheet (step 703). After the first and second polymer sheets are properly aligned and/or offset, the second polymer sheet is stacked onto the first polymer sheet (step 704). Then the first and second polymer sheets are coupled together (step 705).

As set forth above, a first polymer sheet (step 701) and a second polymer sheet are provided (step 702). In some implementations, the first and the second polymer sheets can be similar to the polymer sheet 100 shown in FIG. 1A. As discussed in relation to FIG. 5, in some implementations, the polymer sheets 100 are created by micro-molding the polymer sheets 100 with an etched Si wafer 502. In other implementations, the micro-pores in the polymer sheets 100 can be created by micro-machining, laser micro-ablating a polymer sheet, by 3D inkjet printing, or by injection molding techniques. In certain implementations, a large polymer sheet is manufactured and the large sheet is cut into a plurality of polymer sheets 100.

The method 700 continues with the offset and/or alignment of features on the polymer sheets (step 703). In some implementations, features, such as pores 105 or struts 106, on two adjacent layers are aligned or offset. For example, in the above example of the polymer scaffold 400, the center pore 401 of each polymer sheet is aligned. As discussed above in relation to FIGS. 2A-2C and 3A-3D, the alignment of successive polymer sheets may include an offset along one or more axes of the pores 105. In the example of the polymer scaffold 230, the struts 106 corresponding to the first axis 101 and second axis 102 are offset from the struts 106 corresponding to the third axis 201 and fourth axis 202. In some implementations, the polymer sheets are aligned using an alignment and coupling device, for example a die bonder or flip-chip bonder. For example, a second polymer sheet may be loaded into the flip-chip bonder above a first polymer sheet or a partially created polymer scaffold (including two or more polymer sheets). Extending a dual objective microscope between the partially created scaffold and the second polymer sheet, the images of the partially created scaffold and the second polymer sheet are superimposed into a single image. Using the superimposed image, the flip-chip bonder can align the polymer sheet and scaffold. In some implementations, the alignment by the flip-chip bonder is semi-automated, with a user aligning the polymers sheet and scaffold with the motorized micro-positing stage of the flip-chip bonder. In other implementations, the alignment is fully automated and the flip-chip bonder aligns the polymer sheet and scaffold by using fiduciary markers of by detecting the struts and/or pores of each layer with computer vision. The semi-automated and fully automated placement of the polymer sheets allow for highly accurate alignment across large portions of the polymer scaffold.

At step 704, the second polymer sheet is stacked onto the first polymer sheet. In certain implementations, the stacking of the polymer sheets is also accomplished through a semi-automated process. In some implementations, the semi-automated process is accomplished with a flip-chip bonder or other such device. For example, continuing the example above, responsive to aligning the second polymer sheet with the first polymer sheet, the second sheet is lowered onto the first sheet with the flip-chip bonder. In some implementations, the second polymer sheet is held in place on the flip-chip bonder by an electrostatic charge built up on the Teflon backing 506 attached to the second polymer sheet. In certain implementations, when the second sheet comes into contact with the first polymer sheet, the static charge is released and the natural adhesion properties of the polymer cause the two polymer sheets to stick together. After placement of a polymer sheet, the Teflon backing 506 is removed from the placed polymer sheet.

Next, the method 700 continues by coupling the first polymer sheet to the second polymer sheet. In some implementations, the coupling of the first polymer sheet to the second polymer sheet is accomplished by heating the polymer sheets and/or applying pressure. In other implementations, the layers our bound together with a molecular film of pre-polymer or another biocompatible adhesive, and in yet other implementations the layers are bound together via electrostatic or ionic interactions.

In some implementations, the above steps 702-705 are repeated until the desired height of the polymer scaffold is achieved. In some implementations, the polymer scaffold consists of between 1 and 50 layers. In other implementations, relatively short polymer scaffolds are combined to create larger polymer scaffolds. Doing so allows for the creation of multi-layer scaffolds with features in dimension orthogonal to the face of the individual polymer sheets. For example, a user may first create a large three-layer polymer scaffold. The user may then segment the large three-layer polymer scaffold into four sections and stack the four sections, thus creating a 12 layer polymer scaffold. In certain implementations, the polymer scaffold or polymer sheets make be cut, trimmed, or pressed into a specific shape. For example, a user may create a polymer scaffold and then punch out a plurality of small disk shaped polymer scaffolds from the original polymer scaffold to form cylindrical polymer scaffolds.

### EXAMPLES

The following illustrative examples provide further detail regarding the manufacture and cellular seeding of the polymer scaffolds descried herein. These specific examples are included merely to illustrate certain aspects and implementations of the present disclosure, and they are not intended to limit the disclosure in any respect. Certain general principles described in the examples, however, may be generally applicable to other aspects or implementations of the disclosure. Any features or implementations described above and below can be combined.

### A. Polymer Sheet Fabrication

FIG. 8 shows images of fabricated polymer scaffolds described above and in relation to FIGS. 2A and 2B. The left images (Column 1) in Rows A-D provide various illustrations of the above described short strut offset. The right images (Column 2) illustrate a long strut offset. As illustrated, Row A shows schematic views of the short strut and long strut offsets, respectively, described in FIGS. 2A and 2B. Row B shows scanning electron microscope images of the pores of each offset configuration. Specifically, the images in Row B illustrate the different through pore shapes created when 70 μm thick polymer sheets with 250 μm by 125 μm pores are offset along their short axis (Row B, Column 1) and when the pores are offset along their long axis (Row B, Column 2). Row 3 shows laser confocal reflectance micrographs, and Row D shows brightfield micrographs of the two configurations. The images in Row D illustrate that the method described above of placing polymer sheets with a flip-chip bonder creates highly regular alignments and offsets over long distances.

The polymer scaffolds of FIG. 8, Row B, were manufactured with an etched mold. The mold was fabricated in a four inch (100 mm) diameter, 1 mm thick silicon wafer. A photoresist adhesion promoter was then applied to the Si wafer by spin-coating at 4000 rpm. A 2.4 μm thick photoresist was then applied to the Si wafer. The resulting Si wafer and photoresist were cleaned with a Plasma Asher and then oven backed for 30 minutes at 110° C. The silicon left exposed by the resist was etched with a Surface Technology Systems Inductively Coupled Plasma Etcher (STS-ICP). The silicon was etched to a depth of about 70 μm. After etching, the photoresist was stripped and the Si wafer rinsed, cleaned and spun dry. The sharp corners, edges, and other roughness was removed with a STS—deep reactive ion etch. After subsequent cleaning, the silicon mold was spin coated with a sacrificial layer of maltose in a two step process (1000 rpm for 10 seconds and 3000 rpm for 30 seconds). The sacrificial layer was hardened by baking for 5 minutes.

PGS pre-polymer was synthesized by reacting glycerol and sebacic acid in a 1:1 molar ratio under heat and vacuum. The pre-polymer was dissolved in ethanol and poured into the silicon mold. The mold was then heated at 110° C. for 30 minutes, and then the polymer was cured with heat (160 ° C.) and vacuum (40 mTorr) for 6 to 8 hours. The resulting polymer sheet was demolded by soaking the polymer sheet in deionized water to dissolve the sacrificial layer. After drying and autoclave-sterilizing the polymer sheet, it was soaked in a serum-containing culture medium for 5 to 8 days prior to seeding with cells.

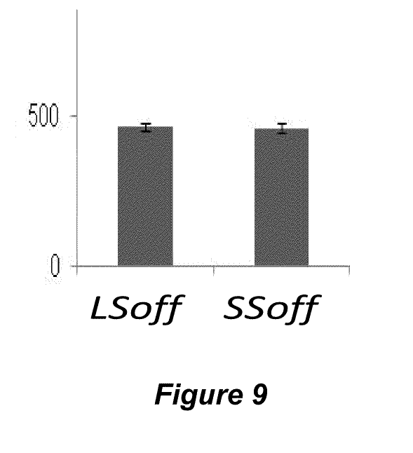

FIG. 9 is a graph illustrating that the two offset configurations have similar effective stiffness measures. The y axis of the graph is the effective stiffness of the scaffold, and two stacking arrangements are shown along the x axis. The first bar, labeled 2L LSoff, indicates the effective stiffness of a two layer scaffold in which the long struts are offset. The second bar, labeled 2L SSoff, indicates the effective stiffness of a two layer scaffold in which the short struts are offset. These data indicate the stiffness of the polymer scaffold does not depend the alignment pattern; instead directional differences in stiffness (anisotropy) are the result of 2D pore shape, where scaffolds are stiffer in the direction of the long axis of the scaffold's rectangular pores.

### B. Cell Seeding

1. Cell Preparation and Polymer Scaffold Construction

For experimentation, two cell types were independently seeded into polymer scaffolds. In a first experiment, heart cells were obtained from 1 to 2 day old neonatal Sprague Dawley rats, according to a protocol approved by an Institute Committee on Animal Care. Briefly, the ventricles were minced, and serially digested using trypsin (0.6 mg/mL) at 4° C. and Type II collagenase (1 mg/mL) at 37° C. The isolated heart cells were counted and used for seeding the scaffold constructs. In the second experiment, cells from the murine myoblast cell line C2C12 were obtained from ATCC (Manassas, Va.) and cultured medium in serum-supplemented Dulbecco's modified Eagle medium.

The polymer scaffolds were manufactured as described above and formed into 5 mm diameter disks and placed in cellular wells. The disks were seeded with either 50,000 C2C12 cells or 3.5 million heart cells/50 μL for every two layers of the polymer scaffold. Upon seeding the cells, 2 mL of medium was added to the well. During the culturing phase the medium was replaced every two days.

2. Cardiac Seeded Scaffolds

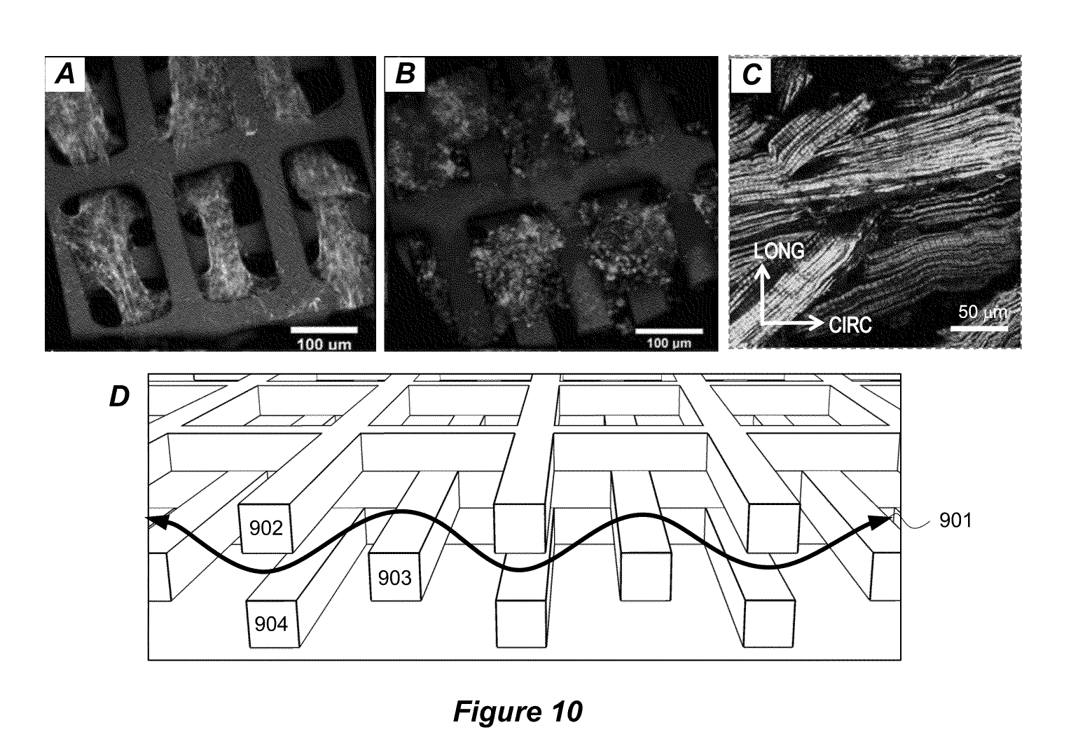

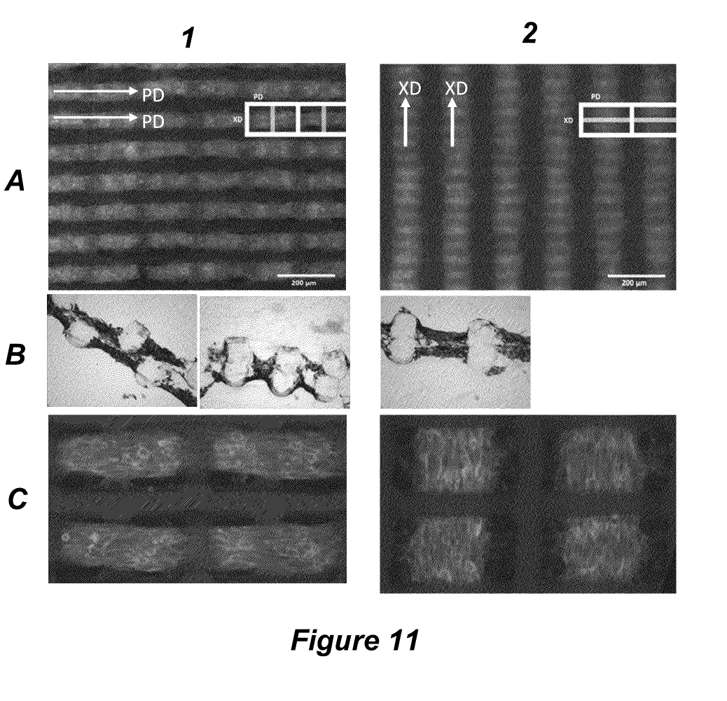

FIGS. 10A and 10B are confocal micrographs of cells within multi-layered degradable polymer scaffolds. The polymer scaffolds in FIGS. 10A and 10B have been seeded with neonatal rat heart cells cultured for 5 days, as described above. FIG. 10A is a confocal micrograph of cells growing on a short strut offset polymer scaffold, such as that of polymer scaffold 200 shown in FIG. 2A and in FIG. 8, Column 1. FIG. 10B is a confocal micrograph of cells growing on a long strut offset polymer scaffold, such as that of polymer scaffold 210 shown in FIG. 2B and in FIG. 8, Column 2. FIG. 10C is a confocal micrograph illustrating the properties of native adult rat cardiac tissue. As shown in FIG. 10C, native cardiac tissue has a high degree of anisotropy as the cells grow predominately along a single axis. FIGS. 10A and 10B show that primary heart cells, when cultured in the tissue engineering scaffold of the disclosure, align in perpendicular directions relative to the 2D rectangular pore shape, similar to the results discussed in the next section in relation to the C2C12 cells of FIG. 11.

In some implementations, the seeded cells stay predominantly in the same plane as the polymer sheets. FIG. 10D is a model generated based on the micrographs of FIG. 10B, and shows a side view of this polymer scaffold, which was also shown in FIG. 2B. The path 901 illustrates the path the rat heart cells in FIG. 10B take through this polymer scaffold. As illustrated in FIG. 10D, and shown in FIG. 10B, the cultured cells predominately weave horizontally through the struts of polymer scaffolds 902 and 903 rather than vertically through the pores within the layers and below scaffold 904. The scaffolds allowed for the growth of muscle fibers, which demonstrated contractile responses to electrical stimulation, tested positive for the cardiac specific sarcomeric α-actinin marker. Combined, these markers indicate heart cell differentiation and functionality similar to in vivo heart cells.

3. C2C12 Seeded Scaffolds

FIG. 11 illustrates C2C12 muscle cell alignment in the two tissue engineering scaffolds shown in Row B of FIG. 8. Column 1 shows short strut offset scaffolds while Column 2 shows long strut offset scaffolds. Row A shows wide view fluorescent micrograph of actin-stained tissue constructs demonstrating macro-alignment of muscle tissue bundles in perpendicular directions relative to the 2D rectangular pore shape. Row B shows hematoxylin and eosin stained cross-sections demonstrating cell seeding throughout the scaffolds. Row C shows confocal micrographs of actin-stained tissue constructs demonstrating alignment of individual cells at the micro-scale.

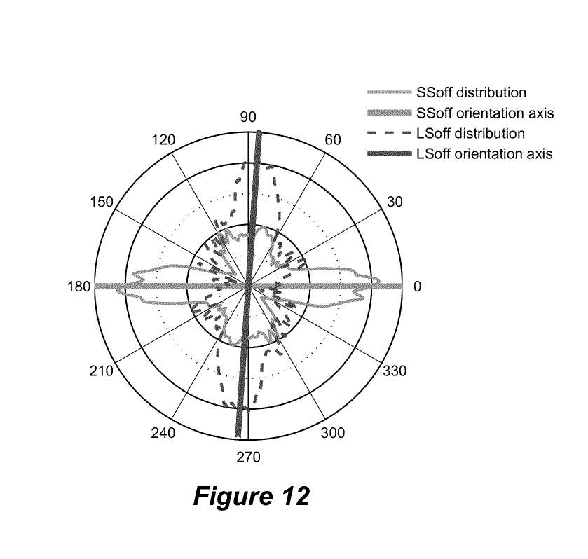

The alignment of the cells in the confocal micrographs of FIG. 11, Row C was quantified via Fast Fourier Transform (FFT) analysis of the confocal micrographs. FIG. 12 is a plot of the FFT analysis. The axis lines represent the predominant alignment direction. The short strut offset (illustrated in blue and corresponding to Column 1 of FIG. 11) is shown to generate a high degree of cellular alignment in the horizontal plane, while long strut offset (illustrated in blue and corresponding to Column 2 of FIG. 11) is shown to generate a high degree of cellular alignment in the vertical plane. Thus, cells seeded in the polymer scaffold develop the highly aligned characteristics of muscle native tissue.

4. Applications

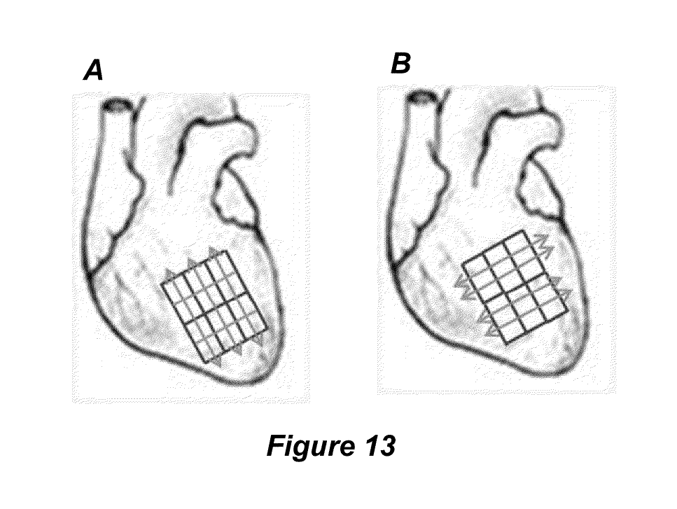

FIGS. 13A and 13B are schematic diagrams showing different example positions for two types of scaffolds as epicardial grafts 1301 and 1302 that provide directional mechanical restraint and aligned cells. The axes of cellular alignment are represented by arrows 1203 and 1204. FIGS. 13A and 13B show how tissue engineering scaffolds with three dimensional structural order, produced as described herein, can be used as epicardial grafts that provide (i) directional mechanical support in the longitudinal direction of the left ventricle, which has been demonstrated to improve systolic function, and (ii) contractility in either the circumferential or longitudinal direction to improve systolic function, in a two-pronged approach to aid in recovery post-myocardial infarction. Similar polymer scaffolds can be used to repair nerve and brain tissue. For example, the scaffold can be used to repair the cerebral fiber pathways of the frontal lobe.

What is claimed:

1. A three-dimensional tissue engineering scaffold device comprising:
a first polymer sheet having a first plurality of micro-scale pores defined therethrough and arranged in a first ordered fashion;
a second polymer sheet having a second plurality of micro-scale pores arranged therethrough and arranged in a second ordered fashion, wherein the second polymer sheet is stacked onto the first polymer sheet such that the first plurality of micro-scale pores are partially aligned with, but are laterally offset from, the second plurality of micro-scale pores, such that the first and second plurality of micro-scale pores define paths through the tissue engineering scaffold.

2. The device of claim 1, wherein the first plurality of micro-scale pores has a first axis that is orthogonal to and shorter than those in a second axis.

3. The device of claim 2, wherein the second plurality of micro-scale pores has a third axis that is orthogonal to and shorter than those in a fourth axis.

4. The device of claim 2, wherein the length of the first plurality of micro-scale pores along the first axis is between about 10 microns and about 500 microns.

5. The device of claim 2, wherein the length of the first plurality of micro-scale pores along the second axis is between about 10 microns and about 500 microns.

6. The device of claim 3, wherein the first axis is parallel to the third axis and the second axis is parallel to the fourth axis.

7. The device of claim 6, wherein the first plurality of micro-scale pores are laterally offset with respect to the second plurality of pores along at least one of the first and second axes.

8. The device of claim 7, wherein the first plurality of micro-scale pores are laterally offset with respect to the second plurality of pores along the first and third axes and are aligned with the second plurality of pores along the second and fourth axes.

9. The device of claim 7, wherein the first plurality of micro-scale pores are laterally offset with respect to the second plurality of pores along the second and fourth axes and are aligned with the second plurality of pores along the first and third axes.

10. The device of claim 7, wherein the first plurality of micro-scale pores are laterally offset with respect to the second plurality of pores along the first and second axes.

11. The device of claim 7, wherein the offset of the first plurality of micro-scale pores from the second plurality of pores creates at least one feature in an axis orthogonal to the surface of the first and second polymer sheets.

12. The device of claim 7, comprising a third polymer sheet coupled to the first and second polymer sheets, wherein the third polymer sheet has a third plurality of micro-scale pores arranged therethrough in a third ordered arrangement, such that the third plurality of micro-scale pores are partially aligned with the second plurality of micro-scale pores such that the first, second, and third pluralities of micro-scale pores form paths through the tissue engineering scaffold.

13. The device of claim 12, wherein the third plurality of micro-scale pores are laterally offset with respect to the second plurality of micro-scale pores along the same axis of the offset between the first plurality of micro-scale pores and the second plurality of micro-scale pores.

14. The device of claim 13, wherein the direction of the offset between the second and third plurality of micro-scale pores along the first axis is opposite to the direction of offset along of the first axis of the second plurality of micro-scale pores with respect to the first plurality of micro-scale pores.

15. The device of claim 13, wherein the direction of the offset between the second and third plurality of micro-scale pores along the first axis is the same direction of the offset along of the first axis of the second plurality of micro-scale pores with respect to the first plurality of micro-scale pores.

16. The device of claim 12, wherein the third plurality of micro-scale pores are laterally offset with respect to the second plurality of micro-scale pores along a different axis than the first plurality of micro-scale pores are offset from the second plurality of micro-scale pores.

17. The device of claim 7, comprising cells seeded in the paths through the tissue engineering scaffold.

18. The device of claim 17, wherein the cells are cardioprogenitor cells.

19. The device of claim 17, wherein the cells comprise at least one of cardiac muscle cells; cardiac fibroblasts; endothelial cells; skeletal muscle cells; smooth muscle cells; endothelial progenitor cells; and skeletal muscle progenitor cells.

20. The device of claim 17, wherein the cells are neuroprogenitor cells.

21. The device of claim 17, wherein the cells comprise at least one of nerve cells; dermal fibroblasts; ectodermal cells; bone cells; cartilage cells; tendon cells; ligament cells; hepatocytes; pancreatic islet cells; intestinal cells; progenitor cells derived from a tissue selected from the group consisting of bone marrow or fat; induced pluripotent stem cells (iPS cells); and genetically transformed cells.

22. The device of claim 17, wherein the pores are configured such that the cells stay predominantly in the same plane as the polymer sheets.

23. The device of claim 1, wherein the first polymer sheet and the second polymer sheet have a height between about 50 microns and about 500 microns.

24. The device of claim 1, wherein the first polymer sheet and the second polymer comprise poly(glycerol sebacate), amino alcohol-based poly(ester amide) (APS), or poly dimethyl siloxane (PDMS).

25. The device of claim 1, wherein the first polymer sheet and the second polymer degradable upon exposure to water, heat, enzymes, or UV light.

26. The device of claim 1, wherein the pores are rectangular, circular, or square in shape.

27. The device of claim 1, wherein the porosity of the first and second polymer sheet is about 60%.

28. The device of claim 1, wherein the first polymer sheet and the second polymer treated with an agent.

29. The device of claim 28, wherein the agent is at least one of solubilized extracellular matrix, collagen, fibronectin, laminin, elastin, an agent that promotes cell adhesion, a cellular growth and/or cell differentiation promoter, growth medium, solubilized extracellular matrix (ECM) or molecular derivative thereof, a fibrosis and/or microbial growth inhibitor, a polymer sheet degradation inhibitor, and a polymer sheet degradation promoter.

30. A method for manufacturing a three-dimensional tissue engineering scaffold device, the method comprising:
providing a first polymer sheet having a first plurality of micro-scale pores defined therethrough and arranged in a first ordered fashion;
providing a second polymer sheet having a second plurality of micro-scale pores defined therethrough and arranged in a second ordered fashion;
stacking the second polymer sheet onto the first polymer sheet such that the first plurality of micro-scale pores are partially aligned with, but are laterally offset from, the second plurality of micro-scale pores, such that the first and second plurality of micro-scale pores define paths through the tissue engineering scaffold; and
bonding the second polymer sheet to the first polymer sheet.

31. The method of claim 30, wherein the first plurality of micro-scale pores has a first axis that is orthogonal to and shorter than those of a second axis.

32. The method of claim 30, wherein the second plurality of micro-scale pores has a third axis that is orthogonal to and shorter than those of a fourth axis.

33. The method of claim 31, wherein the length of the first plurality of micro-scale pores along the first axis is between about 10 microns and about 500 microns.

34. The method of claim 31, wherein the length of the first plurality of micro-scale pores along the second axis is between about 10 microns and about 500 microns.

35. The method of claim 31, wherein the method further comprises laterally offsetting the first plurality of micro-scale pores with respect to the second plurality of pores along at least one of the first and second axes.

36. The method of claim 35, wherein the method further comprises laterally offsetting the first plurality of micro-scale pores with respect to the second plurality of pores along the first and third axes and are aligned with the second plurality of pores along the second and fourth axes.

37. The method of claim 35, wherein the method further comprises laterally offsetting the first plurality of micro-scale pores with respect to the second plurality of pores along the second and fourth axes and are aligned with the second plurality of pores along the first and third axes.

38. The method of claim 35, wherein the method further comprises laterally offsetting the first plurality of micro-scale pores with respect to the second plurality of pores along the first and second axes.

39. The method of claim 30, further comprising:
providing a third polymer sheet having a third plurality of micro-scale pores defined therethrough and arranged in a third ordered fashion;
coupling the third polymer sheet to the first and second polymer sheets such that the third plurality of micro-scale pores are partially aligned with the second plurality of micro-scale pore such that the first, second, and third pluralities of micro-scale pores form paths through the tissue engineering scaffold.

40. The method of claim 30, further comprising:
forming the first polymer sheet on a first sacrificial layer atop a first substrate;
forming the second polymer sheet on a second sacrificial layer atop a second substrate;
removing the first polymer sheet from the first substrate by dissolving the first sacrificial layer; and
removing the second polymer sheet from the second substrate by dissolving second sacrificial layer.

41. The method of claim 30, wherein the first and second sacrificial layers comprise maltose.

42. The method of claim 30, wherein coupling the second polymer sheet to the first polymer sheet further comprises applying heat and pressure to the first and second polymer sheets.

43. The method of claim 30, wherein the first and second polymer sheet comprise poly(glycerol sebacate).

44. The method of claim 30, further comprising seeding cells in the paths through the tissue engineering scaffold.

45. The method of claim 44, wherein the cells are cardioprogenitor cells or heart cells.

46. The device of claim 44, wherein the cells are neuroprogenitor cells or nerve cells.

47. The method of claim 30, further comprising seeding cells in the tissue engineering scaffold such that they align parallel to the aligned axes of the micro-scale pores and weave above the offset axes of the first polymer sheet and below the offset axes of the second polymer sheet, such that the cells stay predominantly in the same plane as the polymer sheets.

48. The method of claim 30, further comprising coating the first and second polymer layer with a growth factor that promotes cell growth.

49. The method of claim 30, wherein the pores are rectangular, circular, or square in shape.

50. A method of treatment, the method comprising:
implanting a tissue engineering scaffold into a patient, wherein the scaffold includes:
a first polymer sheet having a first plurality of micro-scale pores defined therethrough and arranged in a first ordered fashion;
a second polymer sheet having a second plurality of micro-scale pores arranged therethrough and arranged in a second ordered fashion, wherein the second polymer sheet is stacked onto the first polymer sheet such that the first plurality of micro-scale pores are partially aligned with, but are laterally offset from, the second plurality of micro-scale pores, such that the first and second plurality of micro-scale pores define paths through the tissue engineering scaffold.

51. The method of claim 50, wherein the method further includes seeding the tissue engineering scaffold with cells prior to implanting.

52. The method of claim 51, wherein the cells are harvested from the patient.
